(19) United States
(12) Patent Application Publication
(10) Pub. No.: US 2012/0179028 A1
(43) Pub. Date: Jul. 12, 2012

(54) SYSTEM AND METHOD FOR DETERMINING BLOOD-BRAIN BARRIER PERMEABILITY TO WATER

(76) Inventors: Peter Caravan, Cambridge, MA (US); Young Ro Kim, Concord, MA (US); A. Gregory Sorensen, Belmont, MA (US)

(21) Appl. No.: 13/230,990

(22) Filed: Sep. 13, 2011

Related U.S. Application Data
(60) Provisional application No. 61/382,343, filed on Sep. 13, 2010.

(51) Int. Cl. A61B 5/055

(52) U.S. Cl. 600/420

(57) ABSTRACT

A method is provided for measuring a permeability of a subject's blood-brain barrier to water. The method includes acquiring, with an magnetic resonance imaging (“MRI”) system, a first T₁ map of the subject over at least a selected region-of-interest (“ROI”) including a brain of the subject and waiting a delay period selected to allow an affect of the contrast agent on the longitudinal relaxation period to change. The method then includes acquiring, after expiration of the delay period and with the MRI system, a second T₁ map of the subject over at least the selected ROI and determining, using the first T₁ map and the second T₁ map, a fractional volume of vascular compartments in the ROI and a permeability surface area product in the ROI. The method includes creating, using the determined fractional volume and permeability surface area product, a map of water exchange rate in the ROI.

### CROSS-REFERENCE TO RELATED APPLICATIONS

This application claims the benefit of, and herein incorporates by reference in its entirety, U.S. Provisional Patent Application Ser. No. 61/382,343, filed on Sep. 13, 2010, and entitled “System and Method for Determining Blood-Brain Barrier Permeability to Water.”

### BACKGROUND OF THE INVENTION

The present invention relates to systems and methods for magnetic resonance imaging (“MRI”) and, more particularly, to systems and methods for using MRI systems to determine the permeability of a blood-brain barrier.

Neurological diseases represent a tremendous cost to society. Early detection of such diseases is often associated with better outcomes. However, in many neurological diseases, diagnosis is only possible once the disease has progressed to an advanced stage. For instance, using imaging methodologies like MRI or x-ray computed tomography (“CT”), many pathologies are only apparent once gross morphological changes occur. For example, brain tumors often affect the blood-brain barrier (“BBB”). One can attempt to utilize such induced changes in the BBB using, for example, MRI systems to diagnose the neurological disease. However, to appreciate the current limitations of traditional diagnostic methods, such as using known MRI techniques, it is necessary to understand the fundamental principles of MRI physics.

When a substance such as human tissue is subjected to a uniform magnetic field, sometimes referred to as a polarizing, or main magnetic field, B₀, the individual magnetic moments of the excited nuclei in the tissue attempt to align with this main magnetic field, but precess about it in random order at their characteristic Larmor frequency. If the substance, or tissue, is subjected to an excitation magnetic field, B₁, that is in the transverse plane and that is near the Larmor frequency of the nuclei, the net aligned magnetic moment, M, of the nuclei may be rotated, or “tipped,” into the transverse plane to produce a net transverse magnetic moment. A signal is emitted by the excited nuclei, or “spins,” after the excitation signal, B₁, is terminated, and this signal may be received and processed to form an image.

In MRI systems, the excited spins induce an oscillating sine wave signal in a receiving coil. The frequency of this signal is near the Larmor frequency, and its initial amplitude, A₀, is determined by the magnitude of the transverse magnetic moment. The amplitude, A, of the emitted nuclear magnetic resonance (“NMR”) signal decays in an exponential fashion with time. The decay constant depends on the homogeneity of the main magnetic field and on the transverse relaxation time, T₂, which is also referred to as the “spin-spin” relaxation time. The T₂ constant is inversely proportional to the exponential rate at which the aligned precession of the spins would dephase after removal of the excitation signal, B₁, in a perfectly homogeneous magnetic field. The practical value of the T₂ constant is that different tissues have different T₂ values and this can be exploited as a means of enhancing the contrast between such tissues.

Another important factor that contributes to the amplitude of the NMR signal is referred to as the spin-lattice relaxation process, which is characterized by the longitudinal relaxation time, T₁. The T₁ constant describes the recovery of the net magnetic moment to its equilibrium value along the axis of magnetic polarization. The T₁ constant for a given tissue is generally longer than the T₂ constant for the same tissue, and is generally much longer in most substances of medical interest. As with the T₂ constant, the difference in T₁ between different tissues can be exploited to provide image contrast.

When utilizing these NMR signals to produce images, magnetic field gradients are employed to spatially encode the signals. Typically, the region to be imaged is scanned by a sequence of measurement cycles in which these gradients vary according to the particular localization method being used. The resulting set of received NMR signals are digitized and processed to reconstruct the image using one of many well known reconstruction techniques.

Considering the clinical knowledge that, for example, brain tumors often result in a disrupted BBB, one can attempt to utilize this disrupted BBB using traditional MRI techniques by administering a contrast agent or media to the subject and, as the contrast agent leaks across the disrupted BBB into the tumor tissue, achieve an enhanced contrast of the tumor in the resulting image. Utilizing such techniques, clinicians can often diagnose diseases such as ischemic stroke; hemorrhagic stroke; demyelinating diseases, such as multiple sclerosis; encephalitis; and other diseases because all of these diseases are associated with a BBB breakdown. On the other hand, in other diseases, such as vascular amyloidosis, the blood vessel walls thicken and the BBB may become more impermeable. Thus, clinicians cannot rely on the above-described imaging technique to diagnose such diseases.

Therefore, it would be desirable to have a system and method for detecting a variety of changes in the BBB, including large and small changes in the permeability and the impermeability of the BBB. Such systems and methods could, in turn, be used to detect a variety of diseases that are associated with changes in the BBB at an earlier stage. In addition, it would be desirable to have clinically useful tools for readily assessing changes in the BBB in human subjects.

### SUMMARY OF THE INVENTION

The present invention overcomes the aforementioned drawbacks by providing a system and method to determine assess changes in the blood-brain barrier (“BBB”) in vivo by assessing the rate of water exchange across the BBB. A method is provided that includes using an MRI system to obtain a T₁ map of a subject's brain, including the major blood vessels, administering a MRI contrast agent that acts to shorten the T₁ of blood, and obtaining another T₁ map of the brain thereafter.

In accordance with one aspect of the invention, a method is provided for measuring a permeability of a subject's blood-brain barrier to water after the administration of a contrast agent configured to dynamically affect a T₁ relaxation period of the subject in vivo. The method includes acquiring, with an magnetic resonance imaging (“MRI”) system, a first T₁ map of the subject over at least a selected region-of-interest (“ROI”) including a brain of the subject and waiting a delay period selected to allow an affect of the contrast agent on the T₁ relaxation period to change. The method then includes acquiring, after expiration of the delay period and with the MRI system, a second T₁ map of the subject over at least the selected ROI and determining in the ROI using the first T₁ map and the second T₁ map, a fractional volume, fᵥ, of vascular compartments in the ROI and a permeability surface area product, PS. The method also includes creating, using the determined fᵥ and PS, a map of water exchange rate in the ROI.

The foregoing and other aspects and advantages of the invention will appear from the following description. In the description, reference is made to the accompanying drawings which form a part hereof, and in which there is shown by way of illustration at least one embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an MRI system that employs the present invention;

FIG. 2 is a block diagram of an RF system that forms part of the MRI system of FIG. 1;

FIG. 3 is a flow chart setting forth the steps of an example of a method for assessing the characteristics of a blood-brain barrier of a subject in vivo, the method being performed using the MRI systems of FIGS. 1 and 2 in accordance with the present invention; and

FIG. 4 is a graph illustrating a simulated ratio of non-exchange to fast exchange versus exchange rates.

### DETAILED DESCRIPTION OF THE INVENTION

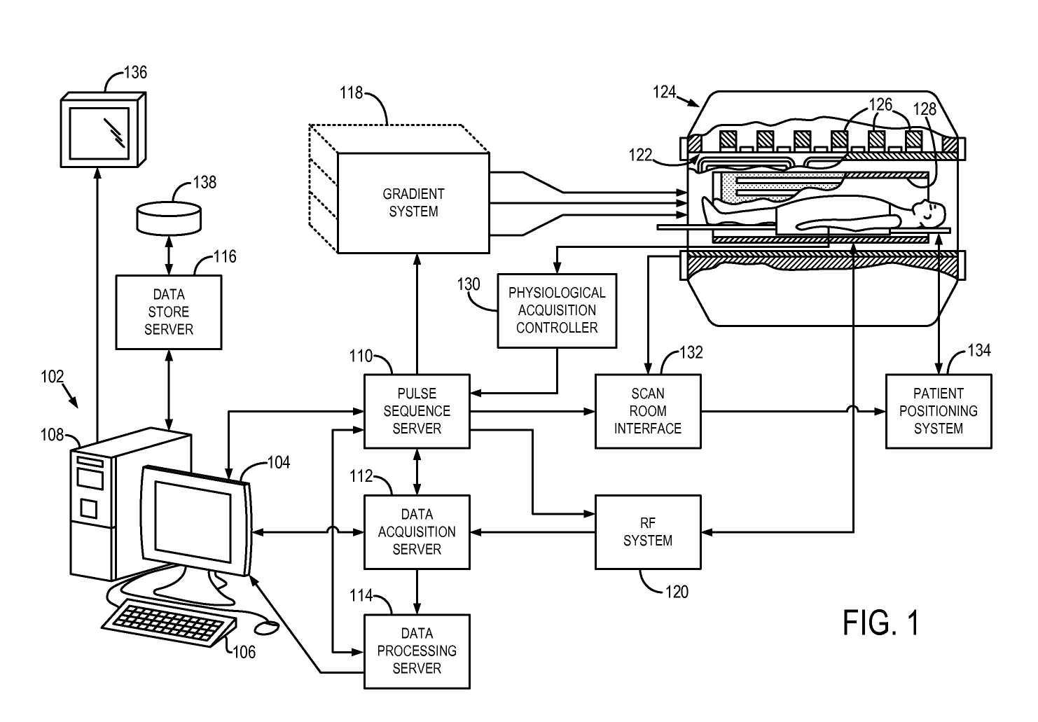

Referring particularly now to FIG. 1, an exemplary magnetic resonance imaging (“MRI”) system 100 is illustrated. The MRI system 100 includes a workstation 102 having a display 104 and a keyboard 106. The workstation 102 includes a processor 108, such as a commercially available programmable machine running a commercially available operating system. The workstation 102 provides the operator interface that enables scan prescriptions to be entered into the MRI system 100. The workstation 102 is coupled to four servers: a pulse sequence server 110; a data acquisition server 112; a data processing server 114; and a data store server 116. The workstation 102 and each server 110, 112, 114, and 116 are connected to communicate with each other.

The pulse sequence server 110 functions in response to instructions downloaded from the workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding MR signals. The gradient coil assembly 122 forms part of a magnet assembly 124 that includes a polarizing magnet 126 and a whole-body RF coil 128.

RF excitation waveforms are applied to the RF coil 128, or a separate local coil (not shown in FIG. 1), by the RF system 120 to perform the prescribed magnetic resonance pulse sequence. Responsive MR signals detected by the RF coil 128, or a separate local coil (not shown in FIG. 1), are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays (not shown in FIG. 1).

The RF system 120 also includes one or more RF receiver channels. Each RF receiver channel includes an RF amplifier that amplifies the MR signal received by the coil 128 to which it is connected, and a detector that detects and digitizes the I and Q quadrature components of the received MR signal. The magnitude of the received MR signal may thus be determined at any sampled point by the square root of the sum of the squares of the I and Q components:

M=\sqrt{I²+Q²} (1);

and the phase of the received MR signal may also be determined:

$$ϕ = {tan}^{- 1} ( \frac{Q}{I} ) . \qquad ( 2 )$$

The pulse sequence server 110 also optionally receives patient data from a physiological acquisition controller 130. The controller 130 receives signals from a number of different sensors connected to the patient, such as electrocardiograph (“ECG”) signals from electrodes, or respiratory signals from a bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processor server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110. Also, navigator signals may be acquired during a scan and used to adjust the operating parameters of the RF system 120 or the gradient system 118, or to control the view order in which k-space is sampled.

The data processing server 114 receives MR data from the data acquisition server 112 and processes it in accordance with instructions downloaded from the workstation 102. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 114 are conveyed back to the workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network to other facilities.

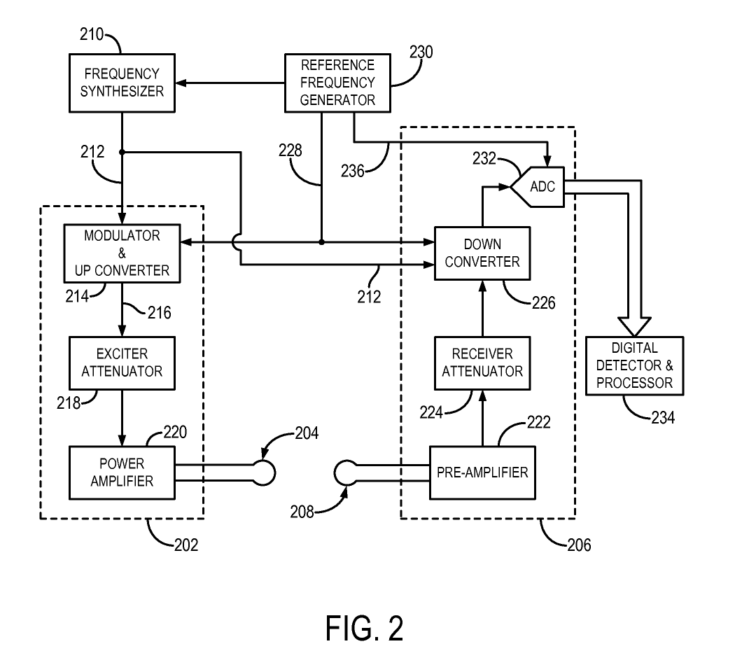

As shown in FIG. 1, the radiofrequency (“RF”) system 120 may be connected to the whole body RF coil 128, or, as shown in FIG. 2, a transmission channel 202 of the RF system 120 may connect to a RF transmission coil 204 and a receiver channel 206 may connect to a separate RF receiver coil 208. Often, the transmission channel 202 is connected to the whole body RF coil 128 and each receiver section is connected to a separate local RF coil.

Referring particularly to FIG. 2, the RF system 120 includes a transmission channel 202 that produces a prescribed RF excitation field. The base, or carrier, frequency of this RF excitation field is produced under control of a frequency synthesizer 210 that receives a set of digital signals from the pulse sequence server 110. These digital signals indicate the frequency and phase of the RF carrier signal produced at an output 212. The RF carrier is applied to a modulator and up converter 214 where its amplitude is modulated in response to a signal, R(t), also received from the pulse sequence server 110. The signal, R(t), defines the envelope of the RF excitation pulse to be produced and is produced by sequentially reading out a series of stored digital values. These stored digital values may be changed to enable any desired RF pulse envelope to be produced.

The magnitude of the RF excitation pulse produced at output 216 is attenuated by an exciter attenuator circuit 218 that receives a digital command from the pulse sequence server 110. The attenuated RF excitation pulses are then applied to a power amplifier 220 that drives the RF transmission coil 204.

The MR signal produced by the subject is picked up by the RF receiver coil 208 and applied through a preamplifier 222 to the input of a receiver attenuator 224. The receiver attenuator 224 further amplifies the signal by an amount determined by a digital attenuation signal received from the pulse sequence server 110. The received signal is at or around the Larmor frequency, and this high frequency signal is down converted in a two step process by a down converter 226. The down converter 226 first mixes the MR signal with the carrier signal on line 212 and then mixes the resulting difference signal with a reference signal on line 228 that is produced by a reference frequency generator 230. The down converted MR signal is applied to the input of an analog-to-digital (“A/D”) converter 232 that samples and digitizes the analog signal. The sampled and digitized signal is then applied to a digital detector and signal processor 234 that produces 16-bit in-phase (I) values and 16-bit quadrature (Q) values corresponding to the received signal. The resulting stream of digitized I and Q values of the received signal are output to the data acquisition server 112. In addition to generating the reference signal on line 228, the reference frequency generator 230 also generates a sampling signal on line 236 that is applied to the A/D converter 232.

When performing imaging techniques that analyze or utilize a change in the blood-brain barrier (“BBB”) to analyze a given pathology, contrast agents are used to shorten T₁ and T₂ relaxation times of water protons in the vascular space. These relaxation times can be represented as T_{1,v} and T_{2,v}, respectively. Whether the contrast agent will have any effect on water in the brain parenchyma will depend on the rate of water exchange, kᵥ, between the vascular compartment and the extravascular compartment (parenchyma). However, there are two limiting cases worth noting. If water exchange between compartments is very fast compared to the longitudinal relaxation rate of the blood, then water in both compartments will be relaxed by the contrast agent. It is noted that the longitudinal relaxation rate is the inverse of longitudinal relaxation time:

$$R - 1 = \frac{1}{{T}_{1}} . \qquad ( 3 )$$

Signal intensity, Sₜᵢₛₛᵤₑ, in the tissue, which includes the vessel and parenchyma, at a given time point for an inversion recovery pulse sequence is given by:

$$\begin{matrix}{S}_{tissue} ( t ) = {M}_{0} ( 1 - 2 {e}^{- t ( \frac{{f}_{v}}{{T}_{1 , v}} + \frac{{f}_{ev}}{{T}_{1 , ev}} )} ) ; \\ and & ( 4 ) \\ {S}_{tissue} ( t ) = {f}_{v} {M}_{0} ( 1 - 2 {e}^{- \frac{t}{{T}_{1 , v}}} ) + {f}_{ev} {M}_{0} ( 1 - 2 {e}^{- \frac{t}{{T}_{1 , ev}}} ) ; & ( 5 )\end{matrix}$$

where fᵥ is the fractional volume of the vascular compartment, fₑᵥ is the fractional volume of the extravascular compartment, fᵥ+fₑᵥ=1, T_{1,v} is the vascular longitudinal relaxation time, and T_{1,ev} the extravascular longitudinal relaxation time. Fast exchange is described by Eqn. (4), and slow exchange is described by Eqn. (5). In the fast exchange limit, relaxation in tissue will be monoexponential. If water exchange is much slower than the rate of relaxation, then the tissue will exhibit bi-exponential relaxation, as shown in Eqn. (5). For the fast exchange case, the fractional blood volume can be estimated by measuring the difference in longitudinal relaxation rates in the tissue before and after contrast agent administration, relative to the longitudinal relaxation rate difference in a blood sample or blood region-of-interest under the same conditions, as follows:

$${f}_{v} = \frac{{R}_{1 , tissue}^{post} - {R}_{1 , tissue}^{pre}}{{R}_{1 , blood}^{post} - {R}_{1 , blood}^{pre}} . \qquad ( 6 )$$

For the slow exchange case, the pre-contrast to post-contrast signal intensity difference in tissue is measured and compared to the signal intensity difference in blood, as follows:

$${f}_{v} = \frac{{S}_{tissue}^{post} - {S}_{tissue}^{pre}}{{S}_{blood}^{post} - {S}_{blood}^{pre}} . \qquad ( 7 )$$

However, if the rate of water exchange is on the order of the relaxation rates observed post-contrast, then the relationship between observed signal, or observed T₁ or R₁ values, and fractional vascular volume becomes more complex. Hazlewood (Biophys J., 1974, 14:583-606) showed that signal is a complicated function of the rates of water exchange between the vascular and extravascular compartments, the respective longitudinal relaxation times in these compartment, and the fractional volumes of these compartments. Donahue et al. (Magn Reson Med, 1996, 36:858-67) showed that applying either Eqn. (6) or Eqn. (7) to systems where there is intermediate water exchange resulted in significant errors in the determination of vascular fractional volume, fᵥ. Applying the fast-exchange model resulted in fᵥ values that were too low, while applying the slow-exchange model resulted in overestimation of fᵥ. Furthermore, different fᵥ values were obtained depending on the longitudinal relaxation rate of the blood post-contrast. This is intuitive because as the longitudinal relaxation rate in the vascular compartment, R_{1,v}, increases then the fast-exchange approximation, kₑₓ>>R_{1,v}, becomes less valid.

Shin et al. (Magn Reson Med, 2006, 56:138-45) used the data and models presented by Donahue and applied it to a study of patients receiving a gadolinium contrast agent. A look-locker echo-planar imaging (“LL-EPI”) sequence was used to determine T₁ values of the brain and blood pool before and after contrast administration. The signal intensity was measured in brain and blood both before and after administration of the contrast agent. Then, the vascular fractional volume was calculated for gray and white matter using Eqns. (6) and (7). This was done for 28 patients and fᵥ versus R_{1,v} was observed for each model in either gray matter or white matter. The aggregate data was fit in a given region with a given model (fast exchange or slow exchange) while iteratively varying both the water exchange rate and the true f to produce a better fit to the observed data. At this point, fᵥ values, which also corresponded to cerebral blood volume (“CBV”) were obtained. These values were reasonable when compared to positron emission tomography (“PET”) studies. Thus, it was proposed that the intravascular-to-extravascular water exchange rate could be estimated to be 0.9 s⁻¹ in white matter and 1.6 s⁻¹ in gray matter. In the study, Shin et al. assumed that CBV and water exchange rate in gray or white matter are the same in all subjects.

Schwarzbauer et al. (Magn Reson Med, 1997, 37:769-77) took an approach similar to that of Donahue. However, the water exchange rates (intravascular-to extravascular, kᵢ, and extravascular-to-intravascular, kₑ) were cast in terms of the permeability surface area product, PS, as follows:

$$\begin{matrix}PS = λ \frac{{k}_{i} {k}_{e}}{{k}_{i} + {k}_{e}} ; & ( 8 ) \\ {k}_{i} = \frac{PS}{{f}_{v}} ; & ( 9 ) \\ {k}_{e} = \frac{PS}{λ - {f}_{v}} ; & ( 10 )\end{matrix}$$

where λ is the tissue-blood partition coefficient defined as the ratio of proton spin densities in tissue and blood; where λ=0.9 for brain.

For tissues like those in the brain where fᵥ is known to be small, Schwarzbauer et al. demonstrated that longitudinal relaxation time, T₁, would appear mono-exponential regardless of the rates of water exchange. Thus, it was shown that this approximation would introduce, at most, approximately five percent in error in the extreme of no water exchange and that this error is diminished by increasing water exchange.

Applying this to Hazlewood's treatment gives the following:

$$\frac{1}{{T}_{1 , tissue}} = \frac{1}{2} ( \frac{1}{{T}_{1 , v}} + \frac{1}{{T}_{1 , ev}} + \frac{PS}{{f}_{v}} + \frac{PS}{λ - {f}_{v}} ) - \frac{1}{2} \sqrt{{( \frac{1}{{T}_{1 , v}} + \frac{PS}{{f}_{v}} - \frac{1}{{T}_{1 , ev}} - \frac{PS}{λ - {f}_{v}} )}^{2} - \frac{4 {PS}^{2}}{{f}_{v} ( λ - {f}_{v} )}} . \qquad ( 11 )$$

Schwarzbauer used a macromolecular contrast agent and gave repeat doses to rats. This allowed the creation of T₁ maps of tissue at different blood T₁ values, T_{1,v}. A non-linear fitting was performed to obtain PS, Fᵥ, and T_{1,v} from a plot of 1/T_{1,tissue} versus 1/T_{1,v}.

Unfortunately, there are two key challenges in determining a water exchange rate that significantly limit these approaches. First, Eqn. (11) includes four parameters that determine the tissue longitudinal relaxation time, T_{1,tissue}: T_{1,v}, T_{1,ev}, PS, and fᵥ. This is partially addressed by measuring T₁ in a region-of-interest (“ROI”) that contains only blood and in an ROI of tissue, such as gray matter, before and after administration of a contrast agent. T_{1,ev} can be estimated from the T₁ of the tissue measured before addition of a contrast agent. T_{1,v} is the T₁ determined in the blood ROI. This leaves two parameters, PS and fᵥ, that contribute to T_{1,tissue} in the presence of a contrast agent. Donahue and Schwarzbauer, working in animal models, addressed this problem by administering an intravascular contrast agent with a very long blood half-life. The long blood half-life allowed for a relatively unchanging steady-state T_{1,v}. They determined T_{1,v} and T_{1,tissue} and then gave more contrast agent to further shorten after which they measured T_{1,v} and T_{1,tissue} again. After collecting several pairs of T_{1,v} and T_{1,tissue} data, they used nonlinear regression to obtain PS and fᵥ. Shin et al. measured a single T_{1,v} and T_{1,tissue} for a number of patients. They then took all the data and determined PS and fᵥ assuming that these parameters were constant for all the subjects in their study.

These approaches are limited in application to clinical subjects. First, the intravascular contrast agents used in the animal studies are, to date, not approved for human use. These authors specified a contrast agent that could provide a constant T_{1,v}. Second, the animal studies required multiple injections or infusions of these contrast agents to change the T_{1,v}. Multiple injections or infusions are time consuming and cumbersome. The approach employed by Shin et al., by virtue of its design, cannot provide water exchange information on a single subject.

The second major challenge in determining water exchange in vivo is the limited dynamic range of T_{1,tissue}. Depending on the water exchange rate and fᵥ, reducing T_{1,v} from 1000 milliseconds to 100 milliseconds may only change T_{1,tissue} by ten percent. In principle, only two measurements of T_{1,tissue} are necessary to obtain fᵥ and PS. However, in practice, with a limited dynamic range in T_{1,tissue} it is useful to obtain more measures of T_{1,tissue} in order to achieve greater confidence in fᵥ and PS.

Another practical issue can arise from T₂* weighting of the tissue signal due to contrast agent injections, which affects measurement accuracy based on the slow exchange case represented by Eqn. (7). In order to provide accurate water exchange quantification, the T₂* effect due to contrast agents is required to be corrected.

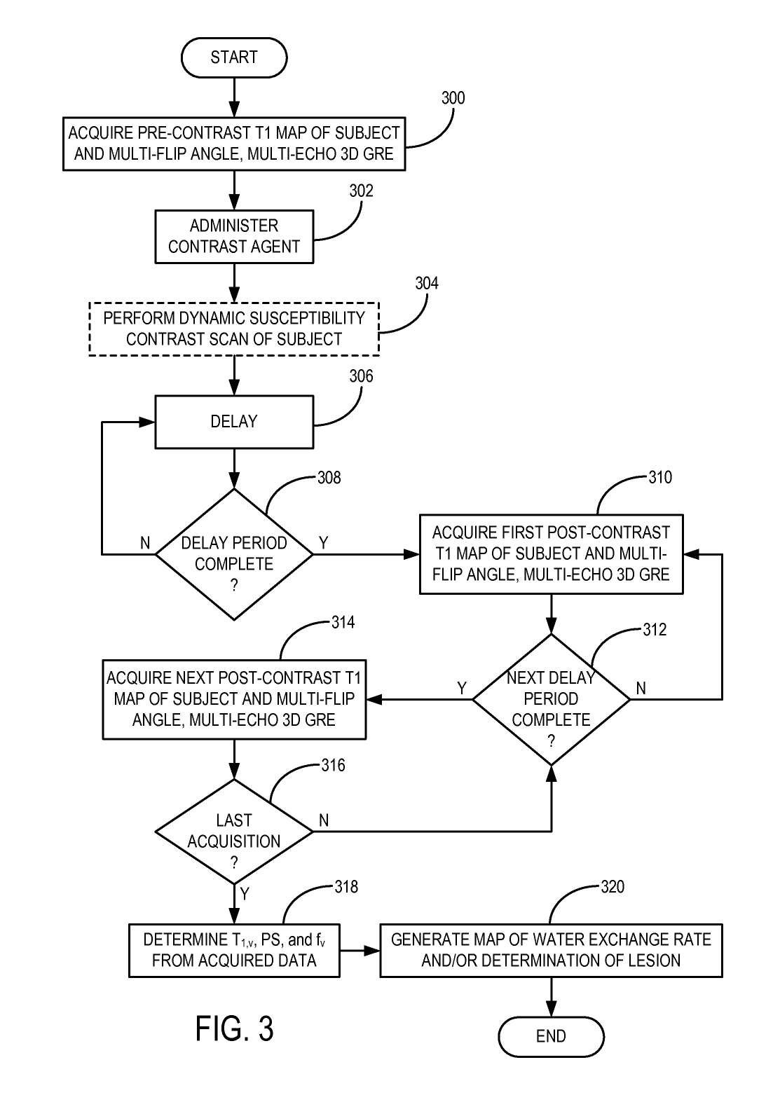

Referring now to FIG. 3, a flow chart setting forth the steps of a method for assessing the permeability of the BBB to water through a determination of water exchange in accordance with the present invention is provided. As will be described, the present invention overcomes the drawbacks of prior art methods to provide a system and method that is clinically applicable to human subjects.

The method begins at process block 300 with the acquisition of a T₁ map of the subject without the presence of any contrast agent in the subject, such as prior to the administration of a contrast agent. Such a T₁ map may be acquired using the above-described MRI system and should, preferably, include the brain of the subject and the major blood vessels therethrough. Thereafter, at process block 302, a contrast agent is administered to the subject. To overcome limitations of the prior art, the present invention may employ only a single dose of a T₁-shortening contrast agent, such as are currently approved for clinical use. It is noted, that process block 300 could be performed at a time after process block 302 and those process blocks following thereafter, but would undesirably require a significant time period between imaging acquisitions to allow the administered contrast agent to be processed by the subject and, thus, the subject be free of the administered contrast agent.

Thereafter, at process block 304, a dynamic susceptibility contrast (“DSC”) scan may be, optionally, acquired immediately after administration of the contrast agent at process block 302. DSC imaging may be used to estimate relative cerebral blood volume and should be done immediately following contrast agent administration, such that signal loss due to the magnetic susceptibility of the contrast agent in the blood is proportional to blood volume. If acquired, the DSC data can be included in the analysis of water exchange in multiple regions-of-interest. That is, the DSC data give the relationship between the blood volume fraction in a first region-of-interest (“ROI”), fᵥ^{A}, and in another ROI, fᵥ^{B}. If multiple regions-of-interest are analyzed simultaneously, then the ratio of fᵥ^{A}:fᵥ^{B}:fᵥ^{C}, and so on, can be fixed from the DSC data. By combining the DSC measurement and the determined blood volume with the T₁ measurements and the determination of blood volume and permeability, increased accuracy of the measurement of water permeability can be achieved.

Following the administration of the contrast agent at process block 302, a delay period is observed at process block 306 to allow the body and, particularly, the subject's brain to uptake the contrast agent. It is noted that the duration of the delay period at process block 306 may be varied to accommodate the optional DSC imaging at process block 304.

As noted, to overcome limitations of the prior art, the present invention may employ only a single dose of a T₁-shortening contrast agent, but can measure multiple values of T_{1,v} and T_{1,tissue} in order to get an accurate measure of PS and fᵥ. In order to sample T_{1,v} at multiple values, as will be described, measurements are made at different points in time after the contrast agent is administered. Thus, after the delay period at process block 306 has been determined to be complete at decision block 308, a first post-contrast T₁ map is acquired from the subject at process block 310. In addition, a multi-flip angle, multi-echo, three-dimensional gradient recalled echo pulse sequence, that will be described, may be used to acquire multiple images at differing flip angles, which reduces the computational burden of the present invention. Again, the data should be acquired from the brain of the subject and include the major blood vessels therethrough and, preferably, be registered with the pre-contrast T₁ map. Thereafter, additional time delays are observed and checked at decision block 312 block and, iteratively, multiple delay periods, T₁ map acquisitions, and multi-echo, three-dimensional gradient recalled echo pulse sequence acquisitions, continue at process block 314, until, at decision block 316, all desired T₁ maps and multi-echo, three-dimensional gradient recalled echo pulse sequence acquisitions have been acquired.

Specifically, after the first post-contrast T₁ map of the brain is obtained, there is, preferably, a delay of 1-120 minutes and then another T₁ map of the brain is acquired. This imaging step may be repeated several times. However, if the optional DSC scan has been performed at process block 304, after contrast agent administration at process block 302 and the DSC imaging at process block 304, the delay is approximately 0-120 minutes following the DSC imaging and, after a second delay of 0-120 minutes, another T₁ map is obtained at process block 314. Additional T₁ maps may also be obtained at later time points, with or without delays between the images.

At process block 318, the values of T_{1,v}, PS, and fᵥ can be determined from the acquired data. Specifically, by acquiring a series of T₁ maps, the value of T_{1,v} will initially be “short” after administration and, as the contrast agent clears, the blood, T_{1,v} will increase. Data for a T₁ map can typically be determined in under a minute by acquiring a series of images with different flip angles in a spoiled gradient recalled echo type sequence or by using a look-locker echo planar imaging (“LL-EPI”) technique. This technique is general to any contrast agent that changes blood T₁ and is not limited to the intravascular agents used in the prior art. The values of PS and fᵥ, and hence water exchange, can be determined from the acquired data using nonlinear regression. Specifically, using Eqns. (6) and (7) from above, the blood volume fraction can be estimated using the fast exchange and slow exchange approximations. The ratio of these measures, f_{v,sx}/f_{v,fx} was found to be proportional to the water exchange rate, as shown below. At process block 320, a map of this ratio f_{v,sx}/f_{v,fx} can be prepared to delineate regional differences in water exchange rate across the brain and identify potential lesions using data fits between the images acquired using the multi-echo, three-dimensional gradient recalled echo pulse sequences.

Additional variations on the above described techniques may include two or more injections of the contrast agent. In some instances it may be advantageous to give multiple administrations of a contrast agent in order to decrease the total time a patient is in the scanner. For instance in order to sample many different values of T_{1,v}, a single dose of the contrast agent can be administered, which will shorten T_{1,v} and then record T₁ maps at different time points. As the contrast agent clears from the blood, T_{1,v} will increase. The time for T_{1,v} to increase to a specific value will be dependent on the pharmacokinetics of the specific contrast agent. An alternate approach is to give a low dose of contrast agent and acquire one or more T₁ maps and then give an additional dose of contrast agent to reduce T_{1,v} even further and obtain additional T₁ maps.

Further still, a T₁ measurement may be made prior to and following contrast agent administration. However, in addition to measuring T₁ in blood and tissue, signal intensity, SI, may be measured in blood and brain tissue. MR signal intensity of brain tissue may be measured using a two-compartment model that considers intravascular and extravascular spaces and uses modified Bloch equations containing proton exchange terms between these compartments. The tissue signal intensity is calculated as a function of the proton, that is water, exchange rate between the intravascular and extravascular compartments and compartment fractions. The apparent blood volume, Vₐₚₚ, is calculated as a function of flip angle, α, assuming no exchange between two compartments as follows:

$${V}_{app}^{α} = \frac{{SI}_{tissue}^{post \text{-} PGC} - {SI}_{tissue}^{pre \text{-} PGC}}{{SI}_{blood}^{post \text{-} PGC} - {SI}_{blood}^{pre \text{-} PGC}} . \qquad ( 12 )$$

The water exchange index, WEI, is a ratio of the apparent blood volume measured with a flip angle of 10 degrees to that measured with a flip angle 90 degrees. Though, for exemplary purposes and simplicity, only two flip angles are described, as will be further detailed, it may be advantageous to use other angle values and use more than two, such as three, angles to improve the ability to detect both abnormally elevated and/or abnormally decreased exchange rates.

As the water exchange rate increases, the extent of Vₐₚₚ overestimation also increases when a low flip angle acquisition is used, but remains approximately constant with a 90 degree flip angle. As such, the ratio of the signal intensities from these two acquisitions provides an indicator of the water exchange rate:

$$WEI = \frac{{V}_{app}^{10 °}}{{V}_{app}^{90 °}} . \qquad ( 13 )$$

The true blood volume fraction, V_{b}, was defined by Vₐₚₚ^{90°}, as V_{b} was demonstrated to be accurately evaluated using a flip angle of approximately 90 degrees. Furthermore, signal intensity simulations were used to examine the WEI as functions of varying repetition time (“TR”), the dose of the intravascular contrast agent, and the fractional volume ratio, V_{b}/Vᵢₜₛₜ, where Vᵢₜₛₜ is the interstitial space volume.

It is noted that water diffusion decreases in both the intracellular and interstitial compartments in hyper-acute stages of stroke, where mixing of protons becomes significantly limited in both compartments. The reduced water diffusion in Vᵢₜₛₜ interferes with the assumption of fast mixing of water molecules, which is adopted by the two-compartment model used for calculating WEI. As such, to properly estimate the WEI independent of rapidly evolving biological milieu, it is desirable to consider the spectrum of biophysical changes during stroke progression in addition to the aforementioned characterization of exchange-affected MR signal.

Also, to avoid inflow effects in the calculation of WEI, as noted above, a three-dimensional pulse sequence is desirable, for example, the listed three-dimensional gradient recalled echo pulse sequence. This can be used to obtain the intravascular SI measurements from, for example, the venous sinuses. WEI corrections were made assuming hypothetical temporal changes in the V_{b}/Vᵢₜₛₜ ratio, based on the time courses of the apparent diffusion coefficient (“ADC”) decay. Because the infarct region WEI may be either over-estimated or under-estimated by the evolving compartment volume differences, as a first pass, it may be assumed that there is a proportional relationship between the effective extravascular space and the relative ADC value. Despite significant changes in Vᵢₜₛₜ, upon correction, factoring in V_{b}(t)/Vᵢₜₛₜ(t) changes did not affect the temporal trend of WEI due to the intrinsically small ipsilesional cerebral V_{b}. However, it can be predicted that when the available blood volume is high, the effect of variable V_{b}(t)/Vᵢₜₛₜ(t) could significantly influence the accuracy of WEI measurements.

In addition, it is noted that, for a spherical compartment, diffusion rates below approximately 0.5×10⁻⁹ m²/s may significantly affect the compartmental water residence time. Analogous to such observations, the residence time of water molecules in the interstitial space may be affected by severe reduction of ipsilesional ADC(t) (approximately 0.3×10⁻⁹ m²/s at t=4 hours). Though the hypothetical increment of diffusion-affected water residence time was not calculated, it is pointed out that the reduced Dᵢₜₛₜ is, in fact, within the range that becomes highly sensitive to τᵢₜₛₜ. The increase in τᵢₜₛₜ can be translated to the reduced water exchange rate, as the mass exchange relationship used in the two-compartment model; that is:

$$\frac{{V}_{itst}}{{τ}_{itst}} = \frac{{V}_{b}}{{τ}_{b}} ; \qquad ( 14 )$$

becomes limited by the reduction in Dᵢₜₛₜ during stroke progression. In fact, the increase in τᵢₜₛₜ directly influences the accuracy of WEI measurements and results in the underestimation of WEI. When diffusion-related τᵢₜₛₜ corrections are applied in addition to the adjustment with V_{b}(t)/Vᵢₜₛₜ(t), the ipsilesional time-dependence of WEI substantially increases, revealing the possibility of progressive blood-brain barrier damage.

Thus, time-dependent hyper-acute changes of various biophysical parameters in a stroke model of permanent middle cerebral artery occlusion can be created. The vascular integrity is distinctively altered within an hour of ischemic onset, concurrently accompanying cytotoxic edema. The two-compartment model using optimizes MRI parameters and the contrast agent dose to achieve measurement accuracy. Furthermore, adjustment of the measured WEI can be made to consider time-dependent biophysical changes, such as reduced diffusion and cellular swelling that may affect the measurement accuracy. By directly examining the movements of water molecules through the vascular membrane, a convenient and practical methodology for assessing blood-brain barrier damage during stroke progression is provided without needing to acquire a leakage profile of extrinsic molecules into the interstitial space, and thereby, to provide a possible predictor of ensuing vasogenic events.

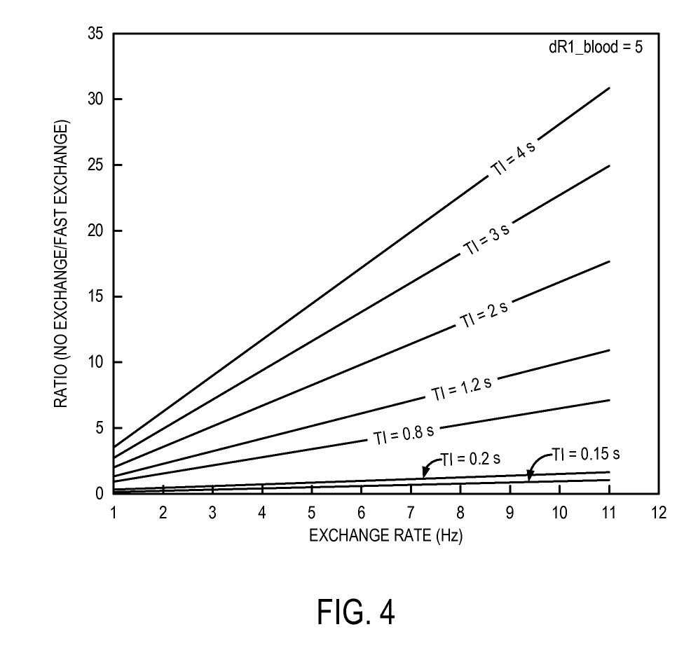

Referring to FIG. 4, simulations have been completed related to the effect of the water exchange rate on the ratio, f_{v,sx}/f_{v,fx}. The simulation was run for different values of the inversion time. T₁ was determined from an inversion recovery sequence where the signal intensity is measured after differing inversion times. The slow exchange fraction volume, f_{v,sx}, was calculated from the signal intensity at any of the inversion times used. For this simulation it is assumed that the change in longitudinal relaxation rate, R₁, of the blood is 5 s⁻¹. This simulation demonstrates that f_{v,sx}/f_{v,fx} increases linearly with increasing exchange rate, such as illustrated in FIG. 4.

Therefore, a method is provided to use a magnetic resonance imaging (“MRI”) system and a contrast agent to determine the permeability of the blood-brain-barrier to water. That is, a method is provided that can measure the rate at which water moves from blood vessels into the brain and vice versa. This permeability will increase in certain pathologies and decrease in others. This method is useful in detection of neuro/psychological disorders and in monitoring how well these diseases respond to treatment. The method involves performing a series of MR scans prior to, and after injection of an approved MRI contrast agent, combining the information from these images to determine the absolute permeability to water in a given brain region and its permeability relative to other brain regions.

The present invention has been described in terms of one or more preferred embodiments, and it should be appreciated that many equivalents, alternatives, variations, and modifications, aside from those expressly stated, are possible and within the scope of the invention.

1. A method for measuring a permeability of a subject's blood brain barrier to water after administration of a contrast agent configured to dynamically affect a longitudinal relaxation time period of the subject in vivo, the method comprising:
a) acquiring, with a magnetic resonance imaging (MRI) system, a first T₁ map of the subject over at least a selected region of interest (ROI) including a brain of the subject;
b) waiting a delay period that is selected to allow an affect of the contrast agent on the longitudinal relaxation time period to change;
c) acquiring, after expiration of the delay period and with the MRI system, a second T₁ map of the subject over at least the selected ROI;
d) determining, using the first T₁ map and the second T₁ map, a fractional volume of vascular compartments in the selected ROI and a permeability surface area product in the ROI; and
e) creating, using the determined fractional volume and permeability surface area product, a map of water exchange rate in the selected ROI.

2. The method of claim 1 further comprising repeating steps b) and c) a plurality of times, thereby creating a plurality of T₁ maps.

3. The method of claim 1 wherein step d) includes determining fractional volume for fast exchange rates by dividing a difference between post-contrast longitudinal relaxation rate in tissue and pre-contrast longitudinal relaxation rate in tissue by a difference between post-contrast longitudinal relaxation rate in blood and pre-contrast longitudinal relaxation rate in blood.

4. The method of claim 1 wherein step d) includes determining fractional volume for slow exchange rates by dividing a difference between post-contrast signal from tissue and pre-contrast signal from tissue by a difference between post-contrast signal from blood and pre-contrast signal from blood

5. The method of claim 1 wherein step e) includes determining a blood volume fraction for a fast exchange rate and for a slow exchange rate, determining a ratio of the blood volume fraction for a fast exchange rate to the blood volume fraction for a slow exchange rate, and mapping the determined ratio across an anatomical image of the selected ROI to create the map of water exchange rate.

6. The method of claim 1 wherein steps a) and c) are completed in less than one minute.

7. The method of claim 1 further comprising, prior to step a), performing a dynamic susceptibility contrast (DSC) scan of the subject.

8. The method of claim 7 wherein step a) includes acquiring the first T₁ map of the subject over a plurality of regions-of-interest and step b) includes acquiring the second T₁ map of the subject over the plurality of regions-of-interest, and wherein step c) includes determining a fractional volume for each region-of-interest in the plurality of regions-of-interest using data acquired from the DSC scan.

9. The method of claim 1 wherein steps a) and c) include acquiring multi-echo signals for each acquisition and creating T₂* maps therefrom.

10. The method of claim 9 wherein step d) includes creating the first T₁ map and the second T₁ map along with T₂* correction.

11. The method of claim 1 wherein steps a) and c) include performing a three-dimensional spoiled gradient echo (SPGR) pulse sequence.

12. The method of claim 11 wherein step d) includes determining a voxel-wise water exchange map illustrating abnormal water exchange regions and regions-of-interest using a flip-angle dependent MRI signal intensity and the first and second T₁ maps.

13. The method of claim 12 wherein step d) includes performing at least one of linear and exponential fits of the flip angle dependent MRI signal intensity to a slow exchange fractional volume, f_{v,sx}, to identify the abnormal water exchange regions.

14. The method of claim 1 further comprising, prior to step a), acquiring a pre-contrast T₁ map of the subject.

15. The method of claim 1 wherein the contrast agent is configured to reduce the longitudinal relaxation time (T₁) period.

16. The method of claim 1 wherein the multi-echo images are collected for adjusting T₂*-associated signal changes.

17. The method of claim 1 wherein the delay period is between 1 and 120 minutes.
